(19) United States
(12) Patent Application Publication
(10) Pub. No.: US 2014/0171358 A1
(43) Pub. Date: Jun. 19, 2014

(54) ANTIMICROBIAL COMPOSITIONS COMPRISING DGLA AND NEOMYCIN SULFATE AND METHODS OF USE THEREOF

(71) Applicant: Dignity Sciences Limited, Dublin (IE)

(72) Inventors: Mehar Manku, Birmingham (GB); John Climax, Dublin (IE); Jonathan Rowe, Waterford, CT (US); David Coughlan, Dublin (IE)

(73) Assignee: Dignity Sciences Limited, Dublin (IE)

(21) Appl. No.: 14/186,200

(22) Filed: Feb. 21, 2014

Related U.S. Application Data
(63) Continuation of application No. 13/478,990, filed on May 23, 2012.
(60) Provisional application No. 61/591,036, filed on Jan. 26, 2012.

(51) Int. Cl. A61K 31/7036; A61K 9/00; A61K 45/06; A61K 31/202

(52) U.S. Cl. CPC A61K 31/7036; A61K 31/202; A61K 9/0014; A61K 9/0053; A61K 45/06
USPC 514/2.8; 514/39; 514/2.9; 514/2.3

(57) ABSTRACT

The present disclosure provides compositions comprising fatty acids, or derivatives thereof (e.g., C1-C4 esters) including, for example, DGLA, 15-OHEPA and/or 15-HETrE, used alone or in combination with one or more antibiotic agents for the treatment of disease and/or disorders such as a skin infection or wound healing.

### PRIORITY CLAIM

This application is a continuation of U.S. patent application Ser. No. 13/478,990, filed on May 23, 2012, which claims the benefit of U.S. Provisional Patent Application Ser. No. 61/591,036, filed Jan. 26, 2012, the entire contents of each of which are incorporated herein by reference.

### FIELD

The disclosure generally relates to compositions comprising fatty acids including, for example, DGLA, 15-OHEPA and/or 15-HETrE, alone or in combination with one or more antibiotic or anti-fungal agents for the treatment of disease and/or disorders such as a skin or gingival infection.

### BACKGROUND

Scores of different microbial species colonize the skin and oral cavity as normal flora. However, when the skin's normal continuity or the oral microenvironment becomes disrupted, the resulting contusions, wounds, lesions, incisions, pockets, lacerations, and/or other disruptions can become infected by a wide variety of microbial species, some of which have become or are rapidly becoming resistant to existing therapies. Accordingly, there exists a need for compositions that are more effective in the treatment of skin and oral infections.

### SUMMARY

The present disclosure provides compositions comprising one or more fatty acids agents including, for example, DGLA, 15-OHEPA and/or 15-HETrE, used alone or in combination with antibiotic agents for the treatment of disease and/or disorders of the skin and gingiva.

The present disclosure also provides methods for treating or preventing skin and gingival infections in a subject in need thereof comprising administering to the subject a pharmaceutical composition comprising a therapeutically effective amount of DGLA, 15-OHEPA, or 15-HETrE or combinations thereof alone or in combination with one or more antibiotic agents. In some embodiments, the pharmaceutical composition comprises about 0.1 wt. % to about 20 wt. % of DGLA, 15-OHEPA, or 15-HETrE.

In some embodiments, the pharmaceutical composition comprises a sub-therapeutic amount of one or more of DGLA, 15-OHEPA, or 15-HETrE along with a therapeutic amount of one or more antibiotic agents. In some embodiments, the pharmaceutical composition comprises a therapeutic amount of one or more of DGLA, 15-OHEPA, or 15-HETrE along with a sub-therapeutic amount of one or more antibiotic agents. In some embodiments, the pharmaceutical composition comprises a sub-therapeutic amount of one or more of DGLA, 15-OHEPA, or 15-HETrE along with a sub-therapeutic amount of one or more antibiotic agents.

In some embodiments, the pharmaceutical composition comprises one or more pharmaceutically acceptable excipients.

In some embodiments, the one or more antibiotic agents are selected from the group consisting of: neomycin sulfate, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, metronidazole, macrolide antibiotics (e.g., clindamycin, erythromycin), quinolone anibiotics (e.g., levofloxacin, ciprofloxacin), penicillins, glycopeptides (e.g., vancomycin), aminoglycosides (e.g., neomycin, gentamicin, tobramycin), trimethoprim/sulfamethoxazole (also known as co-trimoxazole or TMP/SMX), doxycycline, triclosan, metronidazole, monocycline and tetracycline.

In some embodiments, the one or more antibiotic agents are selected from the group consisting of: neomycin salts (e.g., neomycin sulfate), polymixin B, and/or bacitracin salts (e.g., bacitracin zinc). In some embodiments, the one or more antibiotic agents are neomycin sulfate, polymyxin B and bacitracin zinc.

In some embodiments, the step of administering comprises topically applying the composition to an area of the skin or gingival afflicted with lesions. As used herein, the term “lesion” refers broadly to any disruption in the normal continuity and function of the skin or gingiva and includes, for example, contusions, wounds, burns, sores, ulcers, scrapes, incisions, lacerations, skin infection, gingivitis and periodontal disease. In some embodiments, the area of the skin afflicted with lesions is washed prior to application of the pharmaceutical composition. In some embodiments, the lesions are inflammatory type and/or non-inflammatory type lesions.

In some embodiments, applying the composition results in about a 10%, 20%, 30%, 40%, 50%, 60%, 70%, 80%, 90% or more reduction in the lesion.

In some embodiments, the lesion is associated with a microbe such as gram positive bacteria, gram negative bacteria or fungi.

In some embodiments, the lesion is associated with one or more of: Staphylococcus spp., Propionibacterium spp., Streptococcus spp, Corynebacterium spp., Porphyromonas spp. Micrococcus spp., Pseudomonas aeruginosa, Pasteurella multocida, Capnocytophaga canimorsus, Bartonella spp., Klebsiella rhinoscleromatis, Helicobacter spp, Aspergillus niger, Aureobasiduim pullulans, Chaetomium globosum, Gliocladium vixens, Penicillum funiculosum, Candida albicans, Saccharomyces cerevisiae and Vibrio vulnificus

In some embodiments, the pharmaceutical composition is administered to the subject once a day, twice a day, or three times a day.

In some embodiments, the pharmaceutical composition is a cream, lotion, gel or emulsion.

In some embodiments, the subject previously exhibited lesions.

The present disclosure also provides methods of treating or preventing a microbial infection (e.g. a skin or gingival infection) in a subject in need thereof comprising administering to the subject a pharmaceutical composition comprising a therapeutically effective amount of DGLA. In some embodiments, the pharmaceutical composition comprises about 0.1% to about 20 wt. % of DGLA.

In some embodiments, the step of administering comprises topically applying the composition to an area of the skin or gingiva afflicted with lesions. In some embodiments, the area of the skin afflicted with lesions is first washed prior to application of the pharmaceutical composition.

In some embodiments, the lesions are inflammatory type and/or non-inflammatory type lesions.

In some embodiments, the composition reduces about 10%, 20%, 30%, 40%, 50%, 60%, 70%, 80%, 90% or more of the lesion.

In some embodiments, the lesion is associated with one or more of: Staphylococcus spp., Propionibacterium spp., Streptococcus spp, Corynebacterium spp., Porphyromonas spp. Micrococcus spp., Pseudomonas aeruginosa, Pasteurella multocida, Capnocytophaga canimorsus, Bartonella spp., Klebsiella rhinoscleromatis, Helicobacter spp., Aspergillus niger, Aureobasiduim pullulans, Chaetomium globosum, Gliocladium vixens, Penicillum funiculosum, Candida albicans, Saccharomyces cerevisiae and Vibrio vulnificus.

In some embodiments, the pharmaceutical composition is administered to the subject once a day, twice a day, or three times a day.

In some embodiments, the pharmaceutical composition is a cream, lotion, gel, rinse, paste or emulsion.

In some embodiments, the subject previously exhibited a lesion.

The present disclosure also provides compositions for use in treating a skin or oral infection (e.g a microbial, bacterial or fungal infection) comprising a therapeutically effective amount of DGLA, 15-OHEPA, or 15-HETrE. In some embodiments, the composition comprises about 0.1% to about 20 wt. % of DGLA, 15-OHEPA, or 15-HETrE. In some embodiments, the therapeutically effective amount is an amount sufficient to kill or eradicate the microbe in one to a plurality of administrations.

The present disclosure also provides methods for treating or preventing microbial (e.g. bacterial or fungal) infection on the skin or gingiva comprising applying to the lesion one or more of DGLA, 15-OHEPA, or 15-HETrE. In some embodiments, the composition comprises about 0.1% to about 20 wt. % of DGLA, 15-OHEPA, or 15-HETrE.

The present disclosure also provides methods for improving the antimicrobial activity of an agent used in the treatment or prevention of skin or gingival infections comprising adding a composition comprising one or more of DGLA, 15-OHEPA, or 15-HETrE to the agent. In some embodiments, the agent used in the treatment or prevention of skin or gingival infections is an antibiotic or antifungal agent. In some embodiments, the composition comprises about 0.1% to about 20 wt. % of DGLA, 15-OHEPA, or 15-HETrE.

The present disclosure also provides methods of inhibiting one or more skin or gingival pathogens including, for example, its reproduction, growth or recolonization, comprising contacting the one or more skin or oral pathogens with a composition comprising DGLA, 15-OHEPA, or 15-HETrE. In some embodiments, the composition comprises about 0.1% to about 20 wt. % of DGLA, 15-OHEPA, or 15-HETrE.

In some embodiments, the methods may further comprise administering to the subject a steroid. In some embodiments, the steroid is a corticosteroid such as hydrocortisone, prednicarbate, fluticasone and derivatives thereof, or mometasone and derivatives thereof.

In some embodiments, the subject is administered the therapeutically effective amount of DGLA, 15-OHEPA, or 15-HETrE, the one or more antibiotic agents, and the steroid concomitantly.

In some embodiments, the pharmaceutical composition comprises about 0.1 wt. % to about 20 wt. % of DGLA, 15-OHEPA, or 15-HETrE.

In some embodiments, the step of administering comprises topically applying the composition to an area afflicted with contusions, wounds, burns, sores, ulcers, scrapes, incisions, lacerations, skin infection, gingivitis or periodontal disease.

In some embodiments, the area afflicted with contusions, wounds, burns, sores, ulcers, scrapes, incisions, lacerations, skin infection, gingivitis or periodontal disease. is first washed prior to application of the pharmaceutical composition.

In some embodiments, the pharmaceutical composition is administered to the subject once a day, twice a day, or three times a day.

In some embodiments, the pharmaceutical compositions described herein are in the form of a cream, lotion, paste, gel, etc.

The present disclosure also provides methods for improving the efficacy of an agent used in the treatment of contusions, wounds, burns, sores, ulcers, scrapes, incisions, lacerations, skin infection, gingivitis or periodontal disease comprising adding a therapeutically effective amount of DGLA, 15-OHEPA, or 15-HETrE to the agent. In some embodiments, the agent is one or more antibiotic agents.

In some embodiments, about 0.1% to about 20 wt. % of DGLA, 15-OHEPA, or 15-HETrE is added to the agent.

The present disclosure also provides methods for reducing the efficacious dose of an agent used in the treatment of contusions, wounds, burns, sores, ulcers, scrapes, incisions, lacerations, skin infection, gingivitis or periodontal disease comprising adding a therapeutically effective amount of DGLA, 15-OHEPA, or 15-HETrE to the agent.

In some embodiments, about 0.1% to about 20 wt. % of DGLA, 15-OHEPA, or 15-HETrE is added to the agent.

These and other embodiments of the invention are described in further detail below.

### BRIEF DESCRIPTION OF THE FIGURE

FIG. 1 depicts measurements obtained to determine the CZOI values for in vitro inhibition of bacterial growth according to one embodiment of the present disclosure.

### DETAILED DESCRIPTION

The present disclosure provides compositions (e.g., pharmaceutical compositions) and formulations that comprise fatty acid agents including, for example, DGLA, 15-OHEPA and/or 15-HETrE alone; or with one or more antibiotic agents, for example, neomycin sulfate, polymyxin B, and/or bacitracin zinc. Such agents have been found to reduce including, inhibit, the growth of bacteria associated with skin and gingival infections such as Staphylococcus spp. (including, for example, S. aureus, S. lugdunensis, S. schleiferi and other coagulase-negative Staphylococcus spp.), Streptococcus spp. (including, for example, β-haemolytic Streptococci, Viridans group Streptococci, non-haemolytic Streptococci, and Streptococcus milleri group), Corynebacterium spp., Bacillus spp. (including, for example, B. anthracis and B. cereus), Acinetobacter spp., Moraxella spp., Peptostreptococcus spp., Propionibacterium spp., (including, for example, P. Acnes), Candida spp., Pseudomonas spp. and other non-fermentative bacilli (including, for example, P. aeruginosa), Dermatophytes, Enterobacteriaceae, Pasturella multocida, Mycobacterium spp., Haemophilus spp., Nocardia spp., Erysipelothrix rhusiopathiae, Vibrio spp., Enterococcus spp., Eikenella corrodens, anaerobes, Corynebacterium spp., Actinomyces spp., and fungal pathogens. Furthermore, the inventors have found that, in many cases, the use of these fatty acid agents in combination with existing antibacterial agents (e.g., nicotinamide, benzoyl peroxide, adapalene, metronidazole, neomycin sulfate, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, macrolide antibiotics (e.g., clindamycin, erythromycin), quinolone anibiotics (e.g., levofloxacin, ciprofloxacin), penicillins, triclosan, monocycline, glycopeptides (e.g., vancomycin), aminoglycosides (e.g., neomycin, gentamicin, tobramycin), trimethoprim/sulfamethoxazole (also known as co-trimoxazole or TMP/SMX), doxycycline and tetracycline) provides additional reduction in the growth of bacteria compared to each agent used singly. Given their capacity to reduce, inhibit and/or prevent, the growth of bacteria, the compositions and formulations disclosed herein may be used in the treatment of disease and/or disorders associated with the growth of bacteria.

The present disclosure provides compositions comprising fatty acids including, for example, DGLA, 15-OHEPA and/or 15-HETrE in free acid or derivative form, used in combination with antibacterial agents including, for example, nicotinamide, benzoyl peroxide, adapalene, metronidazole, neomycin sulfate, polymyxin B, and bacitracin zinc and triclosan. In some embodiments, the compositions comprise about 0.1 wt. % to about 20 wt. % of DGLA, 15-OHEPA, or 15-HETrE or derivative thereof. Contemplated combinations include, without limitation, DGLA and neomycin sulfate; 15-OHEPA and neomycin sulfate; 15 HETrE and neomycin sulfate; DGLA and polymyxin B; 15-OHEPA and polymyxin B; 15 HETrE and polymyxin B; DGLA and bacitracin zinc; 15-OHEPA and bacitracin zinc; and 15-HETrE and bacitracin zinc; DGLA, neomycin sulfate, polymyxin B and bacitracin zinc; 15-OHEPA neomycin sulfate, polymyxin B and bacitracin zinc; and 15 HETrE neomycin sulfate, polymyxin B and bacitracin zinc. In some embodiments, a composition comprising DGLA, 15-OHEPA and/or 15-HETrE includes a therapeutically effective amount of neomycin sulfate. In some embodiments, a composition comprising DGLA, 15-OHEPA and/or 15-HETrE includes a therapeutically effective amount of polymyxin B. In some embodiments, a composition comprising DGLA, 15-OHEPA and/or 15-HETrE includes a therapeutically effective amount of bacitracin zinc.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

Dihomo-gamma-linolenic acid, also known as cis-8,11,14-eicosatrienoic acid or C 20:3ω6 (“DGLA”), is the elongation product of gamma-linolenic acid, also referred to as gamoleic acid or C 18:3ω6 (“GLA”). GLA is a component of natural oils from a variety of plants such as Echium, blackcurrant, borage, evening primrose, hackelia, trichodesma, and buglossoides, to name a few. As used herein, the term “DGLA” refers to DGLA free acid (e.g., cis-8,11,14-eicosatrienoic acid) and/or a pharmaceutically acceptable ester, derivative, conjugate or salt thereof, or mixtures of any of the foregoing. In some embodiments, DGLA is in the form of a C₁₋₄ alkyl ester such as methyl ester or ethyl ester form.

15-Hydroxy-eicosa-5,8,11,13,17-pentaenoic acid (“15-OHEPA”) is a derivative of EPA. As used herein, the term “15-OHEPA” refers to 15-OHEPA in its free acid form (e.g, 15-hydroxy-eicosa-5,8,11,13,17-pentaenoic acid) and/or a pharmaceutically acceptable ester, derivative, conjugate or salt thereof, or mixtures of any of the foregoing. In some embodiments, the 15-OHEPA is in the form of a C₁₋₄ alkyl ester such as methyl ester or ethyl ester form.

15-Hydroxy-eicosa-8(Z),11(Z),13 (E)-trienoic acid (“15-HETrE”) is a derivative of DGLA. As used herein, the term “15-HETrE” refers to 15-HETrE in its free acid form (e.g., 15-hydroxy-eicosa-8(Z),11(Z),13(E)-trienoic acid) and/or a pharmaceutically acceptable ester, derivative, conjugate or salt thereof, or mixtures of any of the foregoing.

As used herein, the terms “DGLA derivative” and “derivative of DGLA” refer to compounds formed from the chemical conversion of DGLA including, without limitation, 15-HETrE, and esters, derivatives, conjugates or salts thereof, or mixtures of any of the foregoing. One of skill in the art will readily recognize from the chemical structure and other properties whether a given compound is a DGLA derivative.

In one embodiment, DGLA, 15-OHEPA, and/or 15-HETrE is deodorized prior to use in a method or composition as disclosed herein. In one embodiment, crude DGLA, 15-OHEPA, and/or 15-HETrE is mixed with silica and charcoal. In one embodiment, the silica and charcoal are in a ratio of about 1:1 to about 50:1, for example about 1:1, about 2:1, about 3:1, about 4:1, about 5:1, about 6:1, about 7:1, about 8:1, about 9:1, about 10:1, about 12:1, about 14:1, about 15:1, about 16:1, about 18:1, about 20:1, about 25:1, about 30:1, about 35:1, about 40:1, about 45:1, or about 50:1. In one embodiment, the ratio of DGLA (or 15-OHEPA or 15-HETrE) to silica/charcoal is about 1:1 to about 50:1, for example about 1:1, about 2:1, about 3:1, about 4:1, about 5:1, about 6:1, about 7:1, about 8:1, about 9:1, about 10:1, about 12:1, about 14:1, about 15:1, about 16:1, about 18:1, about 20:1, about 25:1, about 30:1, about 35:1, about 40:1, about 45:1, or about 50:1. In one embodiment, crude DGLA, 15-OHEPA, and/or 15-HETrE has been deodorized by filtering over a CELITE filter. In another embodiment, lecithin is used in the deodorizing of the fatty acids.

In various embodiments, the invention provides pharmaceutical compositions, for example topically deliverable compositions, comprising one or more of DGLA, 15-OHEPA, 15-HETrE or mixtures thereof.

In one embodiment, the present disclosure provides pharmaceutical compositions comprising, for example, an amount (e.g., a therapeutically effective amount) of DGLA, 15-OHEPA, 15-HETrE, or a combination thereof. In one embodiment, the pharmaceutical composition comprises about 0.1 wt. % to about 20 wt. % of the DGLA, 15-OHEPA, 15-HETrE, or a combination thereof, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15 wt. %, about 15.1 wt. %, about 15.2 wt. %, about 15.3 wt. %, about 15.4 wt. %, about 15.5 wt. %, about 15.6 wt. %, about 15.7 wt. %, about 15.8 wt. %, about 15.9 wt. %, about 16 wt. %, about 16.1 wt. %, about 16.2 wt. %, about 16.3 wt. %, about 16.4 wt. %, about 16.5 wt. %, about 16.6 wt. %, about 16.7 wt. %, about 16.8 wt. %, about 16.9 wt. %, about 17 wt. %, about 17.1 wt. %, about 17.2 wt. %, about 17.3 wt. %, about 17.4 wt. %, about 17.5 wt. %, about 17.6 wt. %, about 17.7 wt. %, about 17.8 wt. %, about 17.9 wt. %, about 18 wt. %, about 18.1 wt. %, about 18.2 wt. %, about 18.3 wt. %, about 18.4 wt. %, about 18.5 wt. %, about 18.6 wt. %, about 18.7 wt. %, about 18.8 wt. %, about 18.9 wt. %, about 19 wt. %, about 19.1 wt. %, about 19.2 wt. %, about 19.3 wt. %, about 19.4 wt. %, about 19.5 wt. %, about 19.6 wt. %, about 19.7 wt. %, about 19.8 wt. %, about 19.9 wt. %, or about 20 wt % of the DGLA, 15-OHEPA, 15-HETrE, or a combination thereof.

In one embodiment, the pharmaceutical composition further comprises an additional active agent. In one embodiment, the pharmaceutical composition comprises an amount of the additional active agent that is less than the generally recognized therapeutically effective amount for that agent. In one embodiment, the pharmaceutical composition comprises an amount of the additional active agent that is equal to or greater than the generally recognized therapeutically effective amount for that agent. In one embodiment, the additional active agent has not previously been recognized as effective in the treatment or prevention of skin or gingival infections. In another embodiment, the additional active agent is approved for use in the treatment or prevention of skin or gingival infections. In one embodiment, the additional active agent is an antibiotic agent.

In one embodiment, the additional active agent is neomycin sulfate (also referred to as (2R,3 S,4R,5R,6R)-5-amino-2-(aminomethyl)-6-[(1R,2R,3S,4R,6S)-4,6-diamino-2-[(2S,3R,4S,5R)-4-[(3R,4R,5S,6S)-3-amino-6-(aminomethyl)-4,5-dihydroxyoxan-2-yl]oxy-3-hydroxy-5-(hydroxymethyl)oxolan-2-yl]oxy-3-hydroxycyclohexyl]oxyoxane-3,4-diol, sulfuric acid). In one embodiment, the pharmaceutical composition comprises an amount of neomycin sulfate that is less than the generally recognized therapeutically effective amount. In one embodiment, the pharmaceutical composition comprises an amount of neomycin sulfate that is equal to or greater than the generally recognized therapeutically effective amount. In one embodiment, the pharmaceutical composition comprises about 0.5 mg to about 10 mg of neomycin sulfate per gram of pharmaceutical composition, for example about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, or about 10 mg of neomycin sulfate per gram of pharmaceutical composition. In one embodiment, the pharmaceutical composition comprises less than about 3.5 mg of neomycin sulfate per gram of pharmaceutical composition. In one embodiment, the pharmaceutical composition comprises about 3.5 mg of neomycin sulfate per gram of pharmaceutical composition. In one embodiment, the pharmaceutical composition comprises more than about 3.5 mg of neomycin sulfate per gram of pharmaceutical composition. In one embodiment, the pharmaceutical composition comprises no neomycin sulfate.

In one embodiment, the additional active agent is polymyxin B, a mixture of polymyxin B1 and polymyxin B2 (also referred to as Aerosporin, PMB, and N-[4-amino-1-[[1-[[4-amino-1-oxo-1-[[6,9,18-tris(2-aminoethyl)-15-benzyl-3-(1-hydroxyethyl)-12-(2-methylpropyl)-2,5,8,11,14,17,20-heptaoxo-1,4,7,10,13,16,19-heptazacyclotricos-21-yl]amino]butan-2-yl]amino]-3-hydroxy-1-oxobutan-2-yl]amino]-1-oxobutan-2-yl]-6-methyloctanamide). In one embodiment, the pharmaceutical composition comprises an amount of polymyxin B that is less than the generally recognized therapeutically effective amount. In one embodiment, the pharmaceutical composition comprises an amount of the polymyxin B that is equal to or greater than the generally recognized therapeutically effective amount. In one embodiment, the pharmaceutical composition comprises about 1,000 units to about 20,000 units of polymyxin B per gram of pharmaceutical composition (about 119 μg to about 2.38 mg of polymyxin B per gram of pharmaceutical composition), for example about 1,000 units, about 1,500 units, about 2,000 units, about 2,500 units, about 3,000 units, about 3,500 units, about 4,000 units, about 4,500 units, about 5,000 units, about 5,500 units, about 6,000 units, about 6,500 units, about 7,000 units, about 7,500 units, about 8,000 units, about 8,500 units, about 9,000 units, about 9,500 units, about 10,000 units, about 10,500 units, about 11,000 units, about 11,500 units, about 12,000 units, about 12,500 units, about 13,000 units, about 13,500 units, about 14,000 units, about 14,500 units, about 15,000 units, about 15,500 units, about 16,000 units, about 16,500 units, about 17,000 units, about 17,500 units, about 18,000 units, about 18,500 units, about 19,000 units, about 19,500 units, or about 20,000 units of polymyxin B per gram of pharmaceutical composition. In one embodiment, the pharmaceutical composition comprises less than about 5,000 units of polymyxin B per gram of pharmaceutical composition. In one embodiment, the pharmaceutical composition comprises less than about 10,000 units of polymyxin B per gram of pharmaceutical composition. In one embodiment, the pharmaceutical composition comprises more than about 5,000 units of polymyxin B per gram of pharmaceutical composition. In one embodiment, the pharmaceutical composition comprises more than about 10,000 units of polymyxin B per gram of pharmaceutical composition. In one embodiment, the pharmaceutical composition comprises about 5,000 units of polymyxin B per gram of pharmaceutical composition. In one embodiment, the pharmaceutical composition comprises about 6,500 units of polymyxin B per gram of pharmaceutical composition. In one embodiment, the pharmaceutical composition comprises about 10,000 units of polymyxin B per gram of pharmaceutical composition. In one embodiment, the pharmaceutical composition comprises no polymyxin B.

In one embodiment, the additional active agent is bacitracin zinc. In one embodiment, the pharmaceutical composition comprises an amount of bacitracin zinc that is less than the generally recognized therapeutically effective amount. In one embodiment, the pharmaceutical composition comprises an amount of the bacitracin zinc that is equal to or greater than the generally recognized therapeutically effective amount. In one embodiment, the pharmaceutical composition comprises about 100 units to about 800 units of bacitracin zinc (about 2.5 mg to about 20 mg of bacitracin zinc per gram of pharmaceutical composition), for example about 100 units, about 125 units, about 150 units, about 175 units, about 200 units, about 225 units, about 250 units, about 275 units, about 300 units, about 325 units, about 350 units, about 375 units, about 400 units, about 425 units, about 450 units, about 475 units, about 500 units, about 525 units, about 550 units, about 575 units, about 600 units, about 625 units, about 650 units, about 675 units, about 700 units, about 725 units, about 750 units, about 775 units, or about 800 units of bacitracin zinc per gram of pharmaceutical composition. In one embodiment, the pharmaceutical composition comprises less than about 400 units of bacitracin zinc per gram of pharmaceutical composition. In one embodiment, the pharmaceutical composition comprises about 400 units of bacitracin zinc per gram of pharmaceutical composition. In one embodiment, the pharmaceutical composition comprises more than about 400 units of bacitracin zinc per gram of pharmaceutical composition. In one embodiment, the pharmaceutical composition comprises less than about 500 units of bacitracin zinc per gram of pharmaceutical composition. In one embodiment, the pharmaceutical composition comprises about 500 units of bacitracin zinc per gram of pharmaceutical composition. In one embodiment, the pharmaceutical composition comprises more than about 500 units of bacitracin zinc per gram of pharmaceutical composition. In one embodiment, the pharmaceutical composition comprises no bacitracin zinc.

In one embodiment, the additional active agents are neomycin sulfate, polymyxin B and bacitracin zinc. In one embodiment, the pharmaceutical composition comprises an amount of one or more of neomycin sulfate, an amount of polymyxin B and/or an amount of bacitracin zinc that is less than the generally recognized therapeutically effective amount. In one embodiment, the pharmaceutical composition comprises an amount of neomycin sulfate, an amount of polymyxin B and an amount of bacitracin zinc that are each less than the generally recognized therapeutically effective amount. In one embodiment, one gram of pharmaceutical composition comprises about 3.5 mg of neomycin sulfate, about 5,000 units of polymyxin B, and about 400 units of bacitracin zinc. In one embodiment, one gram of pharmaceutical composition comprises about 3.5 mg of neomycin sulfate, about 10,000 units of polymyxin B, and about 500 units of bacitracin zinc. In one embodiment, one gram of pharmaceutical composition comprises about 3.5 mg of neomycin sulfate, about 10,000 units of polymyxin B, and no bacitracin zinc.

In one embodiment, the additional active ingredient is triclosan. In one embodiment the pharmaceutical composition comprises 0.1-1% triclosan, by weight or by volume. In another embodiment the pharmaceutical composition comprises an amount of triclosan that is less than the generally recognized therapeutically effective amount.

In one embodiment, the pharmaceutical composition further comprises an analgesic agent. In one embodiment, the analgesic agent is a topical or systemic analgesic. In one embodiment, the analgesic agent is a topical or systemic analgesic selected from the group consisting of: ibuprofen, diclofenac, capsaicin, lidocaine, and pramoxine HCl.

In one embodiment, the analgesic agent is pramoxine HCl (also referred to as pramocaine HCl, INN, or BAN). In one embodiment, the pharmaceutical composition comprises an amount of pramoxine HCl that is less than the generally recognized therapeutically effective amount. In one embodiment, the pharmaceutical composition comprises an amount of pramoxine HCl that is equal to or greater than the generally recognized therapeutically effective amount. In one embodiment, the pharmaceutical composition comprises about 1 mg to about 20 mg of pramoxine HCl per gram of pharmaceutical composition, for example about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg of pramoxine HCl per gram of pharmaceutical composition.

In one embodiment, one gram of pharmaceutical composition comprises about 3.5 mg of neomycin sulfate, about 10,000 units of polymyxin B, about 500 units of bacitracin zinc, and about 1 mg to about 20 mg of pramoxine HCl. In one embodiment, one gram of pharmaceutical composition comprises about 3.5 mg of neomycin sulfate, about 10,000 units of polymyxin B, about 500 units of bacitracin zinc, and about 1 mg to less than about 10 mg of pramoxine HCl. In one embodiment, one gram of pharmaceutical composition comprises about 3.5 mg of neomycin sulfate, about 10,000 units of polymyxin B, about 500 units of bacitracin zinc, and about 10 mg of pramoxine HCl. In one embodiment, one gram of pharmaceutical composition comprises about 3.5 mg of neomycin sulfate, about 10,000 units of polymyxin B, no bacitracin zinc, and about 1 mg to about 20 mg of pramoxine HCl. In one embodiment, one gram of pharmaceutical composition comprises about 3.5 mg of neomycin sulfate, about 10,000 units of polymyxin B, no bacitracin zinc, and about 1 mg to less than about 10 mg of pramoxine HCl. In one embodiment, one gram of pharmaceutical composition comprises about 3.5 mg of neomycin sulfate, about 10,000 units of polymyxin B, no bacitracin zinc, and about 10 mg of pramoxine HCl.

Any pharmaceutically acceptable excipient known to those of skill in the art may be used in pharmaceutical compositions according to the present disclosure. Any excipient selected for use in the therapeutic and cosmetic compositions should be pharmaceutically and/or cosmetically acceptable and appropriate for the form in which the therapeutic composition will be used, e.g., cream, gel, milk, oil, lotion, paste and the like. Preferably, the excipient has an affinity for the skin or gingiva, is well tolerated, and stable when used in an amount adequate to provide the desired consistency and ease of application. By way of example only, a pharmaceutical composition according to the present disclosure may comprise one or more of: surfactants, preservatives, flavouring agents, co-solvents, viscosity aids, suspension aids, and lipophilic phases. In one embodiment, the pharmaceutical composition comprises excipients suitable for an orally deliverable composition or for a tooth paste. In another embodiment, the composition comprises one or more of: calcium phosphate, cocoa butter, cottonseed oil, fluoride, hydroxyapatite nanocrystals, olive oil, sodium pyruvate, sugar alcohols (e.g. glycerol, sorbitol, xylitol), surfactants (e.g. sodium lauryl sulfate or other detergents) vitamin E, white petrolatum, emulsifying wax, methylparaben, mineral oil, poloxamer 188, propylene glycol, zinc chloride, and purified water. In one embodiment, the pharmaceutical composition comprises cocoa butter, cottonseed oil, olive oil, sodium pyruvate, sodium triplyphosphate, vitamin E, and white petrolatum. In one embodiment, the pharmaceutical composition comprises white petrolatum. In one embodiment, the pharmaceutical composition comprises emulsifying wax, methylparaben, mineral oil, poloxamer 188, propylene glycol, purified water, and white petrolatum.

In one embodiment, the pharmaceutical composition comprises about 0.5 wt. % to about 5 wt. % of a surfactant such as an ethoxylated natural fatty alcohol (e.g., Steareth-2), for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the surfactant. In one embodiment the surfactant is Steareth-2 (e.g., BRIJ S2, Croda International plc).

In one embodiment, the pharmaceutical composition comprises about 0.5 wt. % to about 5 wt. % of an emulsifier such as a polyoxyethylene fatty ether (e.g., Steareth-21), for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the emulsifier. In one embodiment the emulsifier is Steareth-21 (e.g., BRIJ S721, Croda International plc).

In one embodiment, the pharmaceutical composition comprises a stabilizer such as a cetyl alcohol or a saturated cetyl alcohol (e.g., cetyl alcohol). In one embodiment, the pharmaceutical composition comprises about 0.1 wt. % to about 5 wt. % of a stabilizer, for example about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.01 wt. %, about 1.02 wt. %, about 1.03 wt. %, about 1.04 wt. %, about 1.05 wt. %, about 1.06 wt. %, about 1.07 wt. %, about 1.08 wt. %, about 1.09 wt. %, about 1.1 wt. %, about 1.11 wt. %, about 1.12 wt. %, about 1.13 wt. %, about 1.14 wt. %, about 1.15 wt. %, about 1.16 wt. %, about 1.17 wt. %, about 1.18 wt. %, about 1.19 wt. %, about 1.2 wt. %, about 1.21 wt. %, about 1.22 wt. %, about 1.23 wt. %, about 1.24 wt. %, about 1.25 wt. %, about 1.26 wt. %, about 1.27 wt. %, about 1.28 wt. %, about 1.29 wt. %, about 1.3 wt. %, about 1.31 wt. %, about 1.32 wt. %, about 1.33 wt. %, about 1.34 wt. %, about 1.35 wt. %, about 1.36 wt. %, about 1.37 wt. %, about 1.38 wt. %, about 1.39 wt. %, about 1.4 wt. %, about 1.41 wt. %, about 1.42 wt. %, about 1.43 wt. %, about 1.44 wt. %, about 1.45 wt. %, about 1.46 wt. %, about 1.47 wt. %, about 1.48 wt. %, about 1.49 wt. %, about 1.5 wt. %, about 1.51 wt. %, about 1.52 wt. %, about 1.53 wt. %, about 1.54 wt. %, about 1.55 wt. %, about 1.56 wt. %, about 1.57 wt. %, about 1.58 wt. %, about 1.59 wt. %, about 1.6 wt. %, about 1.61 wt. %, about 1.62 wt. %, about 1.63 wt. %, about 1.64 wt. %, about 1.65 wt. %, about 1.66 wt. %, about 1.67 wt. %, about 1.68 wt. %, about 1.69 wt. %, about 1.7 wt. %, about 1.71 wt. %, about 1.72 wt. %, about 1.73 wt. %, about 1.74 wt. %, about 1.75 wt. %, about 1.76 wt. %, about 1.77 wt. %, about 1.78 wt. %, about 1.79 wt. %, about 1.8 wt. %, about 1.81 wt. %, about 1.82 wt. %, about 1.83 wt. %, about 1.84 wt. %, about 1.85 wt. %, about 1.86 wt. %, about 1.87 wt. %, about 1.88 wt. %, about 1.89 wt. %, about 1.9 wt. %, about 1.91 wt. %, about 1.92 wt. %, about 1.93 wt. %, about 1.94 wt. %, about 1.95 wt. %, about 1.96 wt. %, about 1.97 wt. %, about 1.98 wt. %, about 1.99 wt. %, about 2 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt % of the stabilizer. In one embodiment, the stabilizer is cetyl alcohol (e.g., Crodacol C95 EP, Croda International plc).

In one embodiment, the pharmaceutical composition comprises one or more antioxidants such as ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

In one embodiment the antioxidant is ascorbyl palmitate. In one embodiment the antioxidant is α-tocopherol. In one embodiment the antioxidant is ascorbic acid. In one embodiment the antioxidant is idebenone. In one embodiment, the antioxidant is ubiquinone. In one embodiment, the antioxidant is ferulic acid. In one embodiment, the antioxidant is coenzyme Q10. In one embodiment, the antioxidant is lycopene. In one embodiment, the antioxidant is green tea. In one embodiment, the antioxidant is catechins In one embodiment, the antioxidant is epigallocatechin 3-gallate (EGCG). In one embodiment, the antioxidant is green tea polyphenols (GTP). In one embodiment, the antioxidant is silymarin. In one embodiment, the antioxidant is coffeeberry. In one embodiment, the antioxidant is resveratrol. In one embodiment, the antioxidant is grape seed. In one embodiment, the antioxidant is pomegranate extracts. In one embodiment, the antioxidant is genisten. In one embodiment, the antioxidant is pycnogenol. In one embodiment, the antioxidant is niacinamide. In one embodiment, the pharmaceutical composition comprises about 0.01 wt. % to about 0.5 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.1 wt. % to about 0.3 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.3 wt. % to about 0.5 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, and niacinamide. In one embodiment, the pharmaceutical composition comprises about 0.45 wt. % of one or more antioxidants selected from the group consisting of ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, COFFEEBERRY, resveratrol, grape seed, pomegranate extracts, genisten, pycnogenol, and niacinamide. In one embodiment, the pharmaceutical composition comprises about 0.05 wt. % of idebenone. In one embodiment, the pharmaceutical composition comprises about 0.05 wt. % to about 1 wt. % of ubiquinone, for example about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ubiquinone. In one embodiment, the pharmaceutical composition comprises about 0.1 wt. % to about 1 wt. % of ferulic acid, for example about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. % of ferulic acid. In one embodiment, the pharmaceutical composition comprises about 0.01 wt. % to about 0.5 wt. % of ascorbyl palmitate, about 0.01 wt. % to about 0.5 wt. % of α-tocopherol, and about 0.01 wt. % to about 0.5 wt. % of ascorbic acid. In one embodiment the pharmaceutical composition comprises about 0.1 wt. % to about 0.3 wt. % of ascorbyl palmitate, about 0.1 wt. % to about 0.3 wt. % of α-tocopherol, and about 0.05 wt. % to about 0.2 wt. % of ascorbic acid. In one embodiment the pharmaceutical composition comprises about 0.2 wt. % of ascorbyl palmitate, about 0.15 wt. % of α-tocopherol, and about 0.1 wt. % of ascorbic acid.

In one embodiment, the pharmaceutical composition comprises one or more emollients such as a fully saturated triglyceride (e.g., medium-chain triglycerides such as Crodamol GTCC, Croda International plc), myristyl myristate, isopropryl palmitate, and glycerin. In one embodiment, the pharmaceutical composition comprises about 0.5 wt. % to about 20 wt. % of an emollient, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15 wt. %, about 15.1 wt. %, about 15.2 wt. %, about 15.3 wt. %, about 15.4 wt. %, about 15.5 wt. %, about 15.6 wt. %, about 15.7 wt. %, about 15.8 wt. %, about 15.9 wt. %, about 16 wt. %, about 16.1 wt. %, about 16.2 wt. %, about 16.3 wt. %, about 16.4 wt. %, about 16.5 wt. %, about 16.6 wt. %, about 16.7 wt. %, about 16.8 wt. %, about 16.9 wt. %, about 17 wt. %, about 17.1 wt. %, about 17.2 wt. %, about 17.3 wt. %, about 17.4 wt. %, about 17.5 wt. %, about 17.6 wt. %, about 17.7 wt. %, about 17.8 wt. %, about 17.9 wt. %, about 18 wt. %, about 18.1 wt. %, about 18.2 wt. %, about 18.3 wt. %, about 18.4 wt. %, about 18.5 wt. %, about 18.6 wt. %, about 18.7 wt. %, about 18.8 wt. %, about 18.9 wt. %, about 19 wt. %, about 19.1 wt. %, about 19.2 wt. %, about 19.3 wt. %, about 19.4 wt. %, about 19.5 wt. %, about 19.6 wt. %, about 19.7 wt. %, about 19.8 wt. %, about 19.9 wt. %, or about 20 wt. % of an emollient. In one embodiment, the pharmaceutical composition comprises about 0.5 wt. % to about 5 wt. % of any one emollient. In one embodiment, the one or more emollients are selected from the group consisting of medium-chain triglycerides (e.g., Crodamol GTCC, Croda International plc), myristyl myristate, isopropryl palmitate, and glycerin.

In one embodiment, the pharmaceutical composition comprises medium-chain triglycerides (e.g., Crodamol GTCC), myristyl myristate, isopropryl palmitate and glycerin in a combined amount of about 0.5 wt. to about 20 wt. %. In one embodiment, the pharmaceutical composition comprises about 0.5 wt. % to about 5 wt. % of medium-chain triglycerides (e.g., Crodamol GTCC), for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of medium-chain triglycerides (e.g., Crodamol GTCC). In one embodiment, the pharmaceutical composition comprises about 0.5 wt. % to about 5 wt. % of myristyl myristate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of myristyl myristate.

In one embodiment, the pharmaceutical composition comprises about 0.5 wt. % to about 8 wt. % of isopropryl palmitate, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, or about 8 wt. % of isopropryl palmitate.

In one embodiment, the pharmaceutical composition comprises about 0.5 wt. % to about 5 wt. % of glycerin, for example about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of glycerin. in one embodiment, the pharmaceutical composition comprises about 2 wt. % of medium-chain triglycerides (e.g., Crodamol GTCC), about 2 wt. % of myristyl myristate (e.g., Crodamol MM, Croda International plc), about 4 wt. % of isopropryl palmitate (e.g., Crodamol IPP, Croda International plc), and about 1 wt. % of glycerin.

In one embodiment, the pharmaceutical composition comprises a preservative such as phenoxyethanol. In one embodiment, the pharmaceutical composition comprises about 0.1 wt. % to about 5 wt. % of a preservative, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of a preservative. In one embodiment, the preservative is phenoxyethanol. In one embodiment, the pharmaceutical composition comprises about 0.5 wt. % to about 5 wt. % of phenoxyethanol. In one embodiment, the pharmaceutical composition comprises about 0.5 wt. % to about 2 wt. % of phenoxyethanol. In one embodiment, the pharmaceutical composition comprises about 1 wt. % of phenoxyethanol.

In one embodiment, the pharmaceutical composition comprises one or more thickeners, such as a cross-linked polymer (e.g., a cross-linked acrylic acid polymer such as carbomer, available commercially as Carbopol ETD2020NF, Lubrizol Corp.), a polysaccharide (e.g., a xanthan gum such as CPKelko's Keltrol 11K). In one embodiment, the pharmaceutical composition comprises about 0.1 wt. % to about 5 wt. % of one or more thickeners, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more thickeners. In one embodiment, the one or more thickeners is one or more of a cross-linked acrylic acid polymer and a polysaccharide. In one embodiment, the one or more thickeners are Carbopol ETD2020NF and Keltrol 11K. In one embodiment, the pharmaceutical composition comprises about 0.1 wt. % to about 5 wt. % of Carbopol ETD2020NF and about 0.1 wt. % to about 5 wt. % of Keltrol 11K. In one embodiment, the pharmaceutical composition comprises about 0.5 wt. % to about 1 wt. % of Carbopol ETD2020NF and about 0.2 wt. % to about 1 wt. % of Keltrol 11K. In one embodiment, the pharmaceutical composition comprises about 0.8 wt. % of Carbopol ETD2020NF and about 0.4 wt. % of Keltrol 11K.

In one embodiment, the pharmaceutical composition comprises one or more texturizers such as a lecithin (e.g., a liquid soy lecithin such as Leciprime 1400 IPM, Cargill, Inc.). In one embodiment, the pharmaceutical composition comprises about 0.1 wt. % to about 5 wt. % of one or more texturizers, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of one or more texturizers. In one embodiment, the one or more texturizers comprise Leciprime 1400 IPM. In one embodiment, the pharmaceutical composition comprises about 0.1 wt. % to about 5 wt. % of Leciprime 1400 IPM. In one embodiment, the pharmaceutical composition comprises about 0.2 wt. % to about 1 wt. % of Leciprime 1400 IPM. In one embodiment, the pharmaceutical composition comprises about 0.5 wt. % of Leciprime 1400 IPM.

In one embodiment, the pharmaceutical composition comprises one or more fragrances. In one embodiment, the pharmaceutical composition comprises about 0.01 wt. % to about 0.5 wt. % of one or more fragrances, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, or about 0.5 wt. % of one or more fragrances.

In one embodiment, the pharmaceutical composition comprises: about 0.5 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 0.5 wt. % to about 5 wt. % of one or more surfactants; about 0.5 wt. % to about 5 wt. % of one or more emulsifiers; about 0.05 wt. % to about 5 wt. % of one or more stabilizers; about 0.01 wt. % to about 2 wt. % of one or more antioxidants; about 0.5 wt. % to about 20 wt. % of one or more emollients; about 0.1 wt. % to about 5 wt. % of one or more preservatives; about 0.1 wt. % to about 5 wt. % of one or more thickeners; about 0.1 wt. % to about 5 wt. % of one or more texturizers; and about 0.01 wt. % to about 0.5 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of one or more surfactants; about 1 wt. % to about 2 wt. % of one or more emulsifiers; about 0.1 wt. % to about 1 wt. % of one or more stabilizers; about 0.1 wt. % to about 1 wt. % of one or more antioxidants; about 5 wt. % to about 15 wt. % of one or more emollients; about 0.5 wt. % to about 2 wt. % of one or more preservatives; about 0.5 wt. % to about 2 wt. % of one or more thickeners; about 0.1 wt. % to about 2 wt. % of one or more texturizers; and about 0.01 wt. % to about 0.1 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1.65 wt. % of one or more surfactants; about 1.35 wt. % of one or more emulsifiers; about 0.5 wt. % of one or more stabilizers; about 0.45 wt. % of one or more antioxidants; about 9 wt. % of one or more emollients; about 1 wt. % of one or more preservatives; about 1.2 wt. % of one or more thickeners; about 0.5 wt. % of one or more texturizers; and about 0.05 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 0.5 wt. % to about 5 wt. % of Steareth-2; about 0.5 wt. % to about 5 wt. % of Steareth-21; about 0.1 wt. % to about 5 wt. % of cetyl alcohol; about 0.01 wt. % to about 2 wt. % of a combination of medium-chain triglycerides, myristyl myristate, isopropryl palmitate, and/or glycerin; about 0.5 wt. % to about 20 wt. % of one or more emollients; about 0.1 wt. % to about 5 wt. % of phenoxyethanol; about 0.1 wt. % to about 5 wt. % of a combination of carbomer and/or xanthan gum; about 0.1 wt. % to about 5 wt. % of liquid soy lecithin; and about 0.01 wt. % to about 0.5 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of Steareth-2; about 1 wt. % to about 2 wt. % of Steareth-21; about 0.1 wt. % to about 1 wt. % of cetyl alcohol; about 0.1 wt. % to about 1 wt. % of a combination of ascorbyl palmitate, α-tocopherol, and ascorbic acid; about 5 wt. % to about 15 wt. % of a combination of medium-chain triglycerides, myristyl myristate, isopropryl palmitate, and/or glycerin; about 0.5 wt. % to about 2 wt. % of phenoxyethanol; about 0.5 wt. % to about 2 wt. % of a combination of carbomer and/or xanthan gum; about 0.1 wt. % to about 2 wt. % of liquid soy lecithin; and about 0.01 wt. % to about 0.1 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1.65 wt. % of Steareth-2; about 1.35 wt. % of Steareth-21; about 0.5 wt. % of cetyl alcohol; about 0.2 wt. % of ascorbyl palmitate; about 0.15 wt. % of α-tocopherol; about 0.1 wt. % of ascorbic acid; about 2 wt. % of medium-chain triglycerides; about 2 wt. % of myristyl myristate; about 4 wt. % of isopropryl palmitate; about 1 wt. % of glycerin; about 1 wt. % of phenoxyethanol, about 0.8 wt. % of carbomer; about 0.4 wt. % of xanthan gum; about 0.5 wt. % of liquid soy lecithin; and about 0.05 wt. % of one or more fragrances.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 0.5 wt. % to about 5 wt. % of BRIJ S2; about 0.5 wt. % to about 5 wt. % of BRIJ S721; about 0.1 wt. % to about 5 wt. % of Crodacol C95 EP; about 0.01 wt. % to about 2 wt. % of a combination of Crodamol GTCC, Crodamol MM, Crodamol IPP, and/or glycerin; about 0.5 wt. % to about 20 wt. % of one or more emollients; about 0.1 wt. % to about 5 wt. % of phenoxyethanol; about 0.1 wt. % to about 5 wt. % of a combination of Carbopol ETD2020NF and/or Keltrol 11K; about 0.1 wt. % to about 5 wt. % of Leciprime 1400 IPM; and about 0.01 wt. % to about 0.5 wt. % of Mild Care 345 fragrance.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of BRIJ S2; about 1 wt. % to about 2 wt. % of BRIJ S721; about 0.1 wt. % to about 1 wt. % of Crodacol C95 EP; about 0.1 wt. % to about 1 wt. % of a combination of ascorbyl palmitate, α-tocopherol, and ascorbic acid; about 5 wt. % to about 15 wt. % of a combination of Crodamol GTCC, Crodamol MM, Crodamol IPP, and/or glycerin; about 0.5 wt. % to about 2 wt. % of phenoxyethanol; about 0.5 wt. % to about 2 wt. % of a combination of Carbopol ETD2020NF and/or Keltrol 11K; about 0.1 wt. % to about 2 wt. % of Leciprime 1400 IPM; and about 0.01 wt. % to about 0.1 wt. % of Mild Care 345 fragrance.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1 wt. % to about 2 wt. % of BRIJ S2; about 1 wt. % to about 2 wt. % of BRIJ S721; about 0.1 wt. % to about 1 wt. % of Crodacol C95 EP; about 0.1 wt. % to about 1 wt. % of a combination of ascorbyl palmitate, α-tocopherol, and ascorbic acid; about 5 wt. % to about 15 wt. % of a combination of Crodamol GTCC, Crodamol MM, Crodamol IPP, and/or glycerin; about 0.5 wt. % to about 2 wt. % of phenoxyethanol; about 0.5 wt. % to about 2 wt. % of a combination of Carbopol ETD2020NF and/or Keltrol 11K; about 0.1 wt. % to about 5 wt. % of Leciprime 1400 IPM; and about 0.01 wt. % to about 0.1 wt. % of Mild Care 345 fragrance.

In one embodiment, the pharmaceutical composition comprises: about 0.1 wt. % to about 20 wt. % of one or more of DGLA, 15-OHEPA, and 15-HETrE; optionally about 0.5 mg to about 10 mg of neomycin sulfate per gram of pharmaceutical composition; optionally about 1,000 units to about 20,000 units of polymyxin B per gram of pharmaceutical composition; optionally about 100 units to about 800 units of bacitracin zinc per gram of pharmaceutical composition; optionally about 1 mg to about 20 mg of pramoxine HCl; about 1.65 wt. % of BRIJ S2; about 1.35 wt. % of BRIJ S721; about 0.5 wt. % of Crodacol C95 EP; about 0.2 wt. % of ascorbyl palmitate; about 0.15 wt. % of α-tocopherol, about 0.1 wt. % of ascorbic acid; about 2 wt. % Crodamol GTCC; about 2 wt. % of Crodamol MM; about 4 wt. % of Crodamol IPP; about 1 wt. % of glycerin; about 1 wt. % of phenoxyethanol, about 0.8 wt. % of Carbopol ETD2020NF; about 0.4 wt. % of Keltrol 11K; about 0.5 wt. % of Leciprime 1400 IPM; and about 0.05 wt. % of Mild Care 345 fragrance.

In one embodiment, the pharmaceutical composition comprises: about 0.1 wt. % to about 20 wt. % of one or more of DGLA, 15-OHEPA, and 15-HETrE; optionally about 0.01% to about 1.0% mg of triclosan in a pharmaceutical composition;

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In one embodiment, a composition including, for example, a pharmaceutical composition, as disclosed herein is formulated as an aerosol, a gel, an ointment, a lotion, a cream, a gel stick, a liniment, a paste or a spray.

Such formulations may be stable and comprise an amount (e.g., a therapeutically effective amount) of DGLA, 15-OHEPA, 15-HETrE in combination with one or more antibacterial agents selected from the group consisting of: nicotinamide, triclosan, metronidazole, neomycin sulfate, polymyxin B and bacitracin zinc.

The present disclosure also provides the disclosed compositions or formulations as a component in a product for use in the treatment of skin or gingival infections. In one embodiment, the product comprises a container and a pharmaceutical composition comprising a therapeutically effective amount of DGLA, 15-OHEPA, 15-HETrE, or a combination thereof. In one embodiment, the pharmaceutical composition comprises from about 0.1 wt. % to about 20 wt. % of DGLA, 15-OHEPA, 15-HETrE, or a combination thereof. In one embodiment, the product comprises a pharmaceutical composition as disclosed herein.

### Pharmacokinetics/Pharmacodynamic

The pharmacokinetics and /or pharmacodynamics of the compositions comprising DGLA, 15-OHEPA, or 15-HETrE as disclosed herein may be determined by any method known in the art.

In an embodiment, the pharmacokinetics of a composition comprising DGLA, 15-OHEPA, or 15-HETrE as disclosed herein may be examined using a skin blister technique (see, e.g., Tope, Dermatol Surg 25:348:52 (1999)) to determine the amount of various constituents of the composition that are absorbed through the skin. In an exemplary method, a defined area of the skin is contacted with one or more doses of the compositions at one or more time intervals. Next, epidermal blisters may be made by application of controlled suction to an area of the skin (see, e.g., Kiistala (1968) J. Investig. Dermatol. 50:129-137; Kiistala, et al. (1964) Lancet 1964:1444-1445; and Schreiner, et al. (1978) Scand. J. Infect. Dis. 14(Suppl.):233-237). Prior to the start of forming a blister on an area of the skin, the area may be hydrated with a warm compress and/or swabbed with 70% isopropanol. Next, a suction apparatus may be placed on the area of the skin and controlled suction applied to with an electric vacuum pump. The vacuum may be increased slowly over a period of time (e.g, 1 min) up to a maximum negative pressure sufficient to form a blister (e.g., 0.3 kg/cm2 (3.104 Pa)). The pressure may be maintained for several hours (e.g., 2 to 3 h) until half-spherical blisters are formed. As soon as the blisters appeared, the vacuum may be released, and the suction chamber apparatus carefully removed without breaking the blister. The blister fluid (e.g., 50-500 μl) may then be aspirated and examined. Samples of blister fluid may be stored at _{—}70° C. until analysis. The concentration of DGLA, 15-OHEPA, or 15-HETrE or other constituents from the disclosed compositions may be determined in blister fluid samples by any method known in the art including, for example, gas chromatography MS (GC/MS), or reverse-phase high-performance liquid chromatography (HPLC).

The compositions comprising DGLA as provided herein deliver DGLA at a mean flux rate of from about 0.1 ng to about 1 mg/cm2/hr at about 2, 4, 6, 8, 12, 24, 48 or 72 hours after administration. The compostions comprising 15-OHEPA as provided herein deliver 15-OHEPA at a mean flux rate of from about 0.1 ng to about 1 mg/cm2/hr at about 2, 4, 6, 8, 12, 24, 48 or 72 hours after administration. The compostions comprising 15-HETrE as provided herein deliver 15-HETrE at a mean flux rate of from about 0.1 ng to about 1 mg/cm2/hr at about 2, 4, 6, 8, 12, 24, 48 or 72 hours after administration.

Methods of Treatment of Diseases and/or Disorders

The compositions and formulations disclosed herein may be used in the prevention and treatment of diseases and/or disorders including, for example, disease and/or disorders of the skin and gingiva such as contusions, wounds, burns, sores, ulcers, scrapes, incisions, lacerations, skin infection, gingivitis or periodontal disease.

Methods are provided herein for treating or preventing skin or gingival infections in a subject in need thereof comprising administering to the subject a pharmaceutical composition comprising an effective amount including, for example, a therapeutically effective amount (e.g., 0.1 wt. % to about 20 wt. %) of DGLA, 15-OHEPA, or 15-HETrE as described herein.

The term “skin infection” herein refers to any disease or disorder of the skin that presents with one or more occurring or reoccurring symptoms such as erythema, warmth, swelling, tenderness, pain, ulcers, lesion(s), nodules, fever, scaling, plaques, papules, pustules, cysts, and the like. Non-limiting examples of skin infections include cellulitis; erysipelas; impetigo; folliculitis; furuncles; carbuncles; secondarily infected dermatoses such as atopic dermatitis, allergic contact dermatitis and psoriasis; secondarily infected traumatic lesions; acne; and other skin disorders associated with infectious pathogens.

In one embodiment, the present disclosure provides a method of treating or preventing a skin or gingival infection in a subject in need thereof. In one embodiment, the method comprises administering to the subject a pharmaceutical composition as disclosed herein, for example a pharmaceutical comprising a therapeutically effective amount of DGLA, 15-OHEPA, 15-HETrE, or a combination thereof, along with one or more antibiotic agents. In one embodiment, the pharmaceutical composition comprises from about 0.1 wt. % to about 20 wt. % of DGLA, 15-OHEPA, 15-HETrE, or a combination thereof.

In one embodiment, the present disclosure provides a method of inhibiting one or more skin or oral pathogens including, for example, its growth, colonization and/or infection in a subject in need thereof. In one embodiment, the method comprises contacting a skin or oral pathogen with a composition as disclosed herein, for example a composition comprising one or more of DGLA, 15-OHEPA, and 15-HETrE and one or more antibiotic agents. In one embodiment, the skin or gingival pathogen is one or more of: Staphylococcus spp. (including, for example, S. aureus, S. lugdunensis, S. schleiferi and other coagulase-negative Staphylococcus spp.), Streptococcus spp. (including, for example, β-haemolytic Streptococci, Viridans group Streptococci, non-haemolytic Streptococci, and Streptococcus milleri group), Corynebacterium spp., Bacillus spp. (including, for example, B. anthracis and B. cereus), Acinetobacter spp., Moraxella spp., Peptostreptococcus spp., Propionibacterium spp.,(including, for example, P. Acnes), Candida spp., Pseudomonas spp. and other non-fermentative bacilli (including, for example, P. aeruginosa), Dermatophytes, Enterobacteriaceae, Pasturella multocida, Mycobacterium spp., Haemophilus spp., Nocardia spp., Erysipelothrix rhusiopathiae, Vibrio spp., Enterococcus spp., Eikenella corrodens, anaerobes, Corynebacterium spp., Actinomyces spp., and/or fungal pathogens. In one embodiment, the composition comprises from about 0.1 wt. % to about 20 wt. % of DGLA, 15-OHEPA, 15-HETrE, or a combination thereof.

In one embodiment, the method further comprises washing an affected area of the skin (and/or to an area of the skin that is generally prone to development of a skin infection) prior to administering the pharmaceutical composition. As used herein, the term “washing” refers generally to any method known to those of skill in the art for cleansing the skin, exfoliating the skin, removing dirt, oil, dead skin cells and the like from the skin, etc.

In one embodiment, the method comprises topically administering the pharmaceutical composition to an area of the skin or oral cavity infected by a pathogen and/or to an area of the skin or gingiva that is generally prone to development of an infection and/or previously had aninfection.

In one embodiment, the method comprises administering a pharmaceutical composition as disclosed herein once per day, twice per day, three times per day, or more than three times per day.

In one embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the treated area of the skin or gingiva comprises about 10%, about 20%, about 30%, about 40%, about 50%, about 60%, about 70%, about 80%, about 90%, or greater than about 90% fewer lesions than before treatment.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as DGLA, 15-OHEPA, and/or 15-HETrE, is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

In another embodiment, the present disclosure provides a method of slowing progression of or promoting regression of a skin or gingival infection in a subject in need thereof, comprising administering to a subject in need thereof one or more compositions as disclosed herein.

In one embodiment, the present disclosure provides a method of reducing or preventing side effects associated with topical administration of neomycin sulfate. Administration of high doses of neomycin sulfate has been associated with redness and irritation of the skin, allergic reactions (e.g., rash, hives, itching, difficult breathing, chest tightness, swelling of the mouth, face, lips or tongue), bloody stool, dizziness, hearing loss, muscle twitching, ringing in the ears, seizures, tingling or numbness of the skin, vaginal irritation or discharge, and stomach pains or cramps. In one embodiment, a method of reducing side effects associated with topical administration of neomycin sulfate comprises discontinuing administration of a first pharmaceutical composition comprising neomycin sulfate and administering to a subject a second pharmaceutical composition as disclosed herein. In one embodiment, the second pharmaceutical composition includes an amount of neomycin sulfate that is less than the amount of neomycin sulfate in the first pharmaceutical composition. In one embodiment, the second pharmaceutical composition includes an amount of neomycin sulfate that is about equal to or equal to the amount of neomycin sulfate in the first pharmaceutical composition. In one embodiment, the second pharmaceutical composition includes an amount of neomycin sulfate that is more than the amount of neomycin sulfate in the first pharmaceutical composition. In one embodiment, the second pharmaceutical composition includes no neomycin sulfate, essentially no neomycin sulfate, or substantially no neomycin sulfate.

In one embodiment, the present disclosure provides a method of reducing or preventing side effects associated with topical administration of polymyxin B. Administration of high doses of polymyxin B has been associated with redness and irritation of the skin, severe allergic reactions (e.g., rash, hives, itching, difficulty breathing, chest tightness, swelling of the mouth, face, lips, or tongue), changes in the amount of urine, changes in hearing or ringing in the ears, dizziness, drowsiness, flushing of the face, loss of coordination, mental or mood changes (e.g., irritability), severe headaches, stiff neck, tingling or numbness in the mouth, hands, or feet, unusual weakness, unusually fast heartbeat, and changes in vision. In one embodiment, a method of reducing side effects associated with topical administration of polymyxin B comprises discontinuing administration of a first pharmaceutical composition comprising polymyxin B and administering to a subject a second pharmaceutical composition as disclosed herein. In one embodiment, the second pharmaceutical composition includes an amount of polymyxin B that is less than the amount of polymyxin B in the first pharmaceutical composition. In one embodiment, the second pharmaceutical composition includes an amount of polymyxin B that is about equal to or equal to the amount of polymyxin B in the first pharmaceutical composition. In one embodiment, the second pharmaceutical composition includes an amount of polymyxin B that is more than the amount of polymyxin B in the first pharmaceutical composition. In one embodiment, the second pharmaceutical composition includes no polymyxin B, essentially no polymyxin B, or substantially no polymyxin B.

In one embodiment, the present disclosure provides a method of reducing or preventing side effects associated with topical administration of bacitracin zinc. Administration of high doses of bacitracin zinc has been associated with redness and irritation of the skin, severe allergic reactions (e.g., rash, hives, itching, difficulty breathing, chest tightness, swelling of the mouth, face, lips, or tongue), changes in vision, continued redness, burning, or itching, eye pain, and secondary infection. In one embodiment, a method of reducing side effects associated with topical administration of bacitracin zinc comprises discontinuing administration of a first pharmaceutical composition comprising bacitracin zinc and administering to a subject a second pharmaceutical composition as disclosed herein. In one embodiment, the second pharmaceutical composition includes an amount of bacitracin zinc that is less than the amount of bacitracin zinc in the first pharmaceutical composition. In one embodiment, the second pharmaceutical composition includes an amount of bacitracin zinc that is about equal to or equal to the amount of bacitracin zinc in the first pharmaceutical composition. In one embodiment, the second pharmaceutical composition includes an amount of bacitracin zinc that is more than the amount of bacitracin zinc in the first pharmaceutical composition. In one embodiment, the second pharmaceutical composition includes no bacitracin zinc, essentially no bacitracin zinc, or substantially no bacitracin zinc.

In one embodiment, the present disclosure provides a method of reducing scarring in at least a portion of a subject's skin. In one embodiment, the method comprises administering to the subject a therapeutically effective amount of a pharmaceutical composition as disclosed herein. In one embodiment, the amount of scarring per square inch for a given affected area of the subject's skin after administration of a pharmaceutical composition as disclosed herein is less than, or substantially less than the amount of scarring present in the same area of skin before administration of a pharmaceutical composition as disclosed herein. In one embodiment, treatment according to the present method results in a 10% reduction, about a 20% reduction, about a 30% reduction, about a 40% reduction, about a 50% reduction, about a 60% reduction, about a 70% reduction, about a 80% reduction, about a 90% reduction, or more than a 90% reduction in scarring for a given area of the subject's skin. In one embodiment, the reduction in scarring occurs within about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months of the initiation of the treatment method.

The present disclosure also provides methods of improving the antimicrobial activity of an agent used in the treatment of acne. The term “antimicrobial agent” includes antibiotics and antifungals. More specifically, “antimicrobial agents” may include neomycin sulfate, polymyxin B, bacitracin zinc, β-lactams (e.g., ampicillin, amoxicillin, imipenem, meropenem), carbapenems, cephalosporins (e.g., cephalexin, cephalothin, cefalozin, cefuroxime, cefotaxime, ceftazidime), fluoroquinolones, oxazolidinones, lincosamides, metronidazole, macrolide antibiotics (e.g., clindamycin, erythromycin), quinolone anibiotics (e.g., levofloxacin, ciprofloxacin), penicillins, glycopeptides (e.g., vancomycin), aminoglycosides (e.g., neomycin, gentamicin, tobramycin), trimethoprim/sulfamethoxazole (also known as co-trimoxazole or TMP/SMX), triclosan, doxycycline and tetracycline. In one embodiment, the method comprises adding a pharmaceutical comprising one or more of DGLA, 15-OHEPA, and 15-HETrE to the agent. In one embodiment, the agent is one in which no previous antimicrobial activity was appreciated. In one embodiment, the pharmaceutical composition is a pharmaceutical composition as disclosed herein, for example a pharmaceutical composition comprising from about 0.1 wt. % to about 20 wt. % of DGLA, 15-OHEPA, 15-HETrE, or a combination thereof.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Effect of Various Compounds on the Growth of P. Acnes

Several compounds including fatty acids such as DGLA, 15-OHEPA, and 15-HETrE were tested to determine their capacity to inhibit the growth of P. acnes. In an exemplary method, an agar dilution method was used to determine the minimum inhibitory concentration (MIC) of each tested compound. Briefly, the agar dilution method involved preparing a series of concentrations of each compound (e.g., nicotinamide, benzoyl peroxide, adapalene, metronidazole, DGLA, 15-OHEPA, and 15-HETrE) in a Reinforced Clostridial Agar (RCA) media that facilitates growth of P. acnes under anaerobic conditions. An inoculum of P. acnes was prepared by incubation of P. acnes for approximately seven days at 35-37° C. to achieve a ≧1.0 OD₆₀₀ inoculum of P. acnes in RCM broth. A portion of this inoculum was then added to the surface of each plate as a 10 μL spot and incubated at 35-37° C. for 72 hours or more. Growth of P. acnes was then observed and compared to control plates in which no compound has been added, and positive inhibition plates prepared with erythromycin. The growth profile of each colony (spot) was characterized as per the following index: (+++) confluent growth (comparable to control); (++) less confluent growth; (+) marked reduction in growth to multiple tiny, single colonies; and (−) no growth present. The growth of P. acnes in the presence of nicotinamide, benzoyl peroxide, adapalene, metronidazole, DGLA, 15-OHEPA, and 15-HETrE was determined with varying concentrations of the compound (see, Table 2). Next, the MIC was determined as the concentration at which a marked reduction (+) occurs in the appearance of growth on the test plate (as per Clinical and Laboratory Standards Institute M11-A7).

TABLE 2
Effect of Various Compounds on Growth of P. Acnes.
Nicotinamide

| Nicotinamide | Growth Index (+++, ++, +, −) | | | |
|---|---|---|---|---|
| [mg/mL] | Plate 1 | Plate 2 | Plate 3 | Avg |
| 1 | +++ | +++ | +++ | +++ |
| 5 | +++ | +++ | +++ | +++ |
| 10 | ++ | ++ | ++ | ++ |
| 15 | − | + | + | + |
| 25 | − | − | − | − |

Adapalene:

| Adapalene | Growth Index (+++, ++, +, −) | | | |
|---|---|---|---|---|
| [mg/mL] | Plate 1 | Plate 2 | Plate 3 | Avg. |
| 0.2 | +++ | +++ | +++ | +++ |
| 0.4 | +++ | +++ | +++ | +++ |
| 0.6 | ++ | ++ | +++ | ++ |
| 0.8 | ++ | ++ | ++ | ++ |

DGLA:

| DGLA | Growth Index (+++, ++, +, −) | | | |
|---|---|---|---|---|
| [mg/mL] | Plate 1 | Plate 2 | Plate 3 | Avg. |
| 0.2 | +++ | +++ | +++ | +++ |
| 0.4 | +++ | +++ | +++ | +++ |
| 0.6 | ++ | − | ++ | + |
| 0.8 | ++ | + | + | + |
| 1.0 | + | + | + | + |

15-HETrE:

| 15-HETrE | Growth Index (+++, ++, +, −) | | | |
|---|---|---|---|---|
| [mg/mL] | Plate 1 | Plate 2 | Plate 3 | Avg. |
| 0.00001 | +++ | +++ | +++ | +++ |
| 0.0001 | +++ | +++ | +++ | +++ |
| 0.001 | +++ | +++ | +++ | +++ |
| 0.01 | ++ | +++ | ++ | ++ |
| 0.05 | − | − | − | − |

Benzoyl Peroxide:

| BPO | Growth Index (+++, ++, +, −) | | | |
|---|---|---|---|---|
| [mg/mL] | Plate 1 | Plate 2 | Plate 3 | Avg |
| 0.2 | +++ | +++ | +++ | +++ |
| 0.4 | +++ | +++ | +++ | +++ |
| 0.6 | ++ | +++ | ++ | ++ |
| 0.8 | + | + | + | + |
| 1.0 | − | − | − | − |

Metronidazole:

| Metronidazole | Growth Index (+++, ++, +, −) | | | |
|---|---|---|---|---|
| [mg/mL] | Plate 1 | Plate 2 | Plate 3 | Avg |
| 0.2 | +++ | +++ | +++ | +++ |
| 0.4 | +++ | +++ | +++ | +++ |
| 0.6 | +++ | +++ | +++ | +++ |
| 0.8 | +++ | +++ | +++ | +++ |
| 1.0 | +++ | ++ | +++ | +++ |

15-OHEPA:

| 15-OHEPA | Growth Index (+++, ++, +, −) | | | |
|---|---|---|---|---|
| [mg/mL] | Plate 1 | Plate 2 | Plate 3 | Avg. |
| 0.010 | +++ | +++ | +++ | +++ |
| 0.025 | +++ | +++ | +++ | +++ |
| 0.050 | +++ | +++ | +++ | +++ |
| 0.075 | + | + | + | + |
| 0.1 | − | − | − | − |

The MIC for DGLA was determined to be >0.4, ≦0.6. Additionally, the MICs for 15-HETrE and 15-OHEPA were determined to be >0.01, ≦0.05 and >0.05, ≦0.075, respectively.

### Example 2

### Effects of Fatty Acid Compounds in Combination with Other Compounds on the Growth of P. Acnes

The compounds tested singly in Example 1 were tested in combination with one another to determine the capacity of the combination to inhibit the growth of P. acnes. In an exemplary method, the MIC was determined for each of the combination as described in Example 1. Briefly, test combinations included DGLA with nicotinamide, benzoyl peroxide, adapalene, or metronidazole; 15-HETrE with nicotinamide, benzoyl peroxide, adapalene, or metronidazole; and 15-OHEPA with nicotinamide, benzoyl peroxide, adapalene, or metronidazole. The tables below show the growth of P. acnes in the presence of 0.4 or 1.0 mg/mL DGLA with varying concentrations of nicotinamide, benzoyl peroxide, adapalene, or metronidazole (Table 3); the growth of P. acnes in the presence of 0.01 or 0.05 mg/mL 15-HETrE with varying concentrations of nicotinamide, benzoyl peroxide, adapalene, or metronidazole (Table 5); and the growth of P. acnes in the presence of 0.5 or 0.1 mg/mL 15-OHEPA with varying concentrations of nicotinamide, benzoyl peroxide, adapalene, or metronidazole (Table 7).

TABLE 3
Combinations of Antibacterial Compositions with DGLA

| Nicotinamide + 0.4 mg/mL dGLA (<MIC): | | | | | Nicotinamide + 1.0 mg/mL dGLA (<MIC): | | | | |
|---|---|---|---|---|---|---|---|---|---|
| Nicotinamide | Growth Index (+++, ++, +, −) | | | | Nicotinamide | Growth Index (+++, ++, +, −) | | | |
| [mg/mL] | Plate 1 | Plate 2 | Plate 3 | Avg. | [mg/mL] | Plate 1 | Plate 2 | Plate 3 | Avg. |
| 1 | +++ | +++ | +++ | +++ | 1 | +++ | +++ | +++ | +++ |
| 5 | +++ | +++ | +++ | +++ | 5 | +++ | +++ | +++ | +++ |
| 10 | +++ | +++ | +++ | +++ | 10 | + | ++ | ++ | ++ |
| 15 | + | + | + | + | 15 | − | − | − | − |
| 25 | − | − | − | − | 25 | − | − | − | − |
| Benzoyl Peroxide + 0.4 mg/mL dGLA (<MIC): | | | | | Benzoyl Peroxide + 1.0 mg/mL dGLA (<MIC): | | | | |
| BPO | Growth Index (+++, ++, +, −) | | | | BPO | Growth Index (+++, ++, +, −) | | | |
| [mg/mL] | Plate 1 | Plate 2 | Plate 3 | Avg. | [mg/mL] | Plate 1 | Plate 2 | Plate 3 | Avg. |
| 0.2 | +++ | +++ | +++ | +++ | 0.2 | ++ | ++ | + | ++ |
| 0.4 | + | − | − | − | 0.4 | − | + | − | − |
| 0.6 | − | − | − | − | 0.6 | − | − | − | − |
| 0.8 | − | − | − | − | 0.8 | − | − | − | − |
| 1.0 | − | − | − | − | 1.0 | − | − | − | − |
| Adapalene + 0.4 mg/mL dGLA (<MIC): | | | | | Adapalene + 1.0 mg/mL dGLA (<MIC): | | | | |
| Adapalene | Growth Index (+++, ++, +, −) | | | | Adapalene | Growth Index (+++, ++, +, −) | | | |
| [mg/mL] | Plate 1 | Plate 2 | Plate 3 | Avg. | [mg/mL] | Plate 1 | Plate 2 | Plate 3 | Avg. |
| 0.2 | +++ | +++ | +++ | +++ | 0.2 | +++ | +++ | +++ | +++ |
| 0.4 | +++ | +++ | +++ | +++ | 0.4 | +++ | +++ | +++ | +++ |
| 0.6 | +++ | +++ | +++ | +++ | 0.6 | +++ | +++ | +++ | +++ |
| 0.8 | +++ | +++ | +++ | +++ | 0.8 | +++ | +++ | +++ | +++ |
| 1.0 | +++ | +++ | +++ | +++ | 1.0 | +++ | +++ | +++ | +++ |
| Metronidazole + 0.4 mg/mL dGLA (<MIC): | | | | | Metronidazole + 1.0 mg/mL dGLA (<MIC): | | | | |
| Metronidazole | Growth Index (+++, ++, +, −) | | | | Metronidazole | Growth Index (+++, ++, +, −) | | | |
| [mg/mL] | Plate 1 | Plate 2 | Plate 3 | Avg. | [mg/mL] | Plate 1 | Plate 2 | Plate 3 | Avg. |
| 0.2 | +++ | +++ | +++ | +++ | 0.2 | +++ | +++ | +++ | +++ |
| 0.4 | +++ | +++ | +++ | +++ | 0.4 | +++ | +++ | +++ | +++ |
| 0.6 | +++ | +++ | +++ | +++ | 0.6 | +++ | +++ | +++ | +++ |
| 0.8 | +++ | ++ | ++ | ++ | 0.8 | +++ | +++ | ++ | +++ |
| 1.0 | ++ | ++ | ++ | ++ | 1.0 | ++ | ++ | ++ | ++ |

DGLA in combination with other compounds including nicotinamide, metranidazole or adapalene did not reduce the growth of P. Acnes below that of DGLA used alone. However, a spike of 0.4 mg/ml DGLA reduced the MIC obtained with benzoyl peroxide from >0.6, ≦0.8 to >0.2, ≦0.4. These results suggest that DGLA and benzoyl peroxide may exhibit synergy since 0.4 mg/ml DGLA has no effect on its own but when it is added to benzoyl peroxide it is able to further decrease the growth rate of P. acnes below that of the DGLA single compound treatment (see, Table 4).

TABLE 4
DGLA Combination Summary

| | | MIC | MIC |
|---|---|---|---|
| | MIC (mg/mL) | (mg/mL) with | (mg/mL) with |
| | as per P- | 0.4 mg/mL | 1.0 mg/mL |
| | 11-0007 or P-11-0008 | dGLA | dGLA |
| Compound | Addendum A | spike** | spike*** |
| Metronidazole | >1 | >1 | >1 |
| Nicotinamide | >10, ≦15 | >10, ≦15 | >10, ≦15 |
| BPO | >0.6, ≦0.8 | >0.2, ≦0.4 | >0.2, ≦0.4 |
| Erythromycin | >0.00001, ≦0.00005 | N/App | N/App |
| dGLA | >0.4, ≦0.6** | N/App | N/App |
| Adapalene | >0.8 | >0.6* | >0.6* |
| 15-HETrE | >0.01, ≦0.05 | N/App | N/App |

*Precipitate observed in plates of 0.8 mg/mL and higher. MIC not achieved.
**dGLA supplied by Sigma, cat# E4504
***dGLA supplied by Cayman, cat# 90230

TABLE 5
Combinations of Antibacterial Compositions with HETrE

| Nicotinamide + 0.01 mg/mL 15-HETrE (<MIC): | | | | | Nicotinamide + 0.05 mg/mL 15-HETrE (>MIC) | | | | |
|---|---|---|---|---|---|---|---|---|---|
| Nicotinamide | Growth Index (+++, ++, +, −) | | | | Nicotinamide | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 1 | +++ | +++ | +++ | +++ | 1 | − | − | − | − |
| 5 | +++ | +++ | +++ | +++ | 5 | − | − | − | − |
| 10 | ++ | ++ | ++ | ++ | 10 | − | − | − | − |
| 15 | − | − | − | − | 15 | − | − | − | − |
| 25 | − | − | − | − | 25 | − | − | − | − |
| Benzoyl Peroxide + 0.01 mg/mL 15-HETrE(<MIC): | | | | | Benzoyl Peroxide + 0.05 mg/mL 15-HETrE(>MIC) | | | | |
| BPO | Growth Index (+++, ++, +, −) | | | | BPO | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | +++ | +++ | +++ | +++ | 0.4 | − | − | − | − |
| 0.6 | ++ | ++ | ++ | ++ | 0.6 | − | − | − | − |
| 0.8 | + | + | + | + | 0.8 | − | − | − | − |
| 1.0 | − | − | − | − | 1.0 | − | − | − | − |
| Adapalene + 0.01 mg/mL 15-HETrE(<MIC): | | | | | Adapalene + 0.05 mg/mL 15-HETrE(>MIC) | | | | |
| Adapalene | Growth Index (+++, ++, +, −) | | | | Adapalene | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | +++ | +++ | +++ | +++ | 0.4 | − | − | − | − |
| 0.6 | ++ | ++ | ++ | ++ | 0.6 | − | − | − | − |
| 0.7 | + | + | + | + | 0.7 | − | − | − | − |
| 0.8 | + | − | − | − | 0.8 | − | − | − | − |
| Metronidazole + 0.01 mg/mL 15-HETrE (<MIC): | | | | | Metronidazole + 0.05 mg/mL 15-HETrE (>MIC) | | | | |
| Metronidazole | Growth Index (+++, ++, +, −) | | | | Metronidazole | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | +++ | +++ | +++ | +++ | 0.4 | − | − | − | − |
| 0.6 | +++ | +++ | +++ | +++ | 0.6 | − | − | − | − |
| 0.8 | +++ | +++ | +++ | +++ | 0.8 | − | − | − | − |
| 1.0 | +++ | +++ | +++ | +++ | 1.0 | − | − | − | − |

15-HETrE in combination with other compounds including nicotinamide, metranidazole or benzoyl peroxide did not reduce the growth of P. Acnes below that of 15-HETrE used alone. However, a spike of 0.01 mg/ml HETrE to adapalene did reduce the MIC of adapalene from >0.8 to >0.6, ≦0.7. These results suggest that 15-HETrE and adapalene may exhibit synergy since 0.01 mg/ml HETrE has no effect on its own but when it is added to adapalene it is able to further decrease the growth rate of P. acnes below that of the 15-HETrE single compound treatment (see, Table 6).

TABLE 6
15-HETrE Combination Summary

| | | MIC | MIC |
|---|---|---|---|
| | MIC (mg/mL) | (mg/mL) with | (mg/mL) with |
| | as per P- | 0.01 mg/mL | 0.05 mg/mL |
| | 11-0007 or P-11-0008 | 15-HETrE | 15-HETrE |
| Compound | Addendum A | spike | spike* |
| Metronidazole | >1 | >1 | <0.2 |
| Nicotinamide | >10, ≦15 | >10, ≦15 | <1 |
| BPO | >0.6, ≦0.8 | >0.6, ≦0.8 | <0.2 |
| Adapalene | >0.8 | >0.6, ≦0.7 | <0.2 |
| 15-HETrE | >0.01, ≦0.05 | N/App | N/App |

*0.05 mg/mL 15-HETrE is an inhibitory concentration so complete inhibition was expected for all plates spiked with 0.05 mg/mL..

TABLE 7
Combinations of Antibacterial Compositions with 15-OHEPA

| Nicotinamide + 0.05 mg/mL 15-OHEPA(<MIC): | | | | | Nicotinamide + 0.1 mg/mL 15-OHEPA(>MIC) | | | | |
|---|---|---|---|---|---|---|---|---|---|
| Nicotinamide | Growth Index (+++, ++, +, −) | | | | Nicotinamide | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 1 | +++ | +++ | +++ | +++ | 1 | − | − | − | − |
| 5 | +++ | +++ | +++ | +++ | 5 | − | − | − | − |
| 10 | +++ | +++ | +++ | +++ | 10 | − | − | − | − |
| 15 | − | − | − | − | 15 | − | − | − | − |
| 25 | − | − | − | − | 25 | − | − | − | − |
| Benzoyl Peroxide + 0.05 mg/mL 15-OHEPA(<MIC): | | | | | Benzoyl Peroxide + 0.1 mg/mL 15-OHEPA(>MIC | | | | |
| BPO | Growth Index (+++, ++, +, −) | | | | BPO | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | ++ | ++ | ++ | ++ | 0.4 | − | − | − | − |
| 0.6 | + | + | + | + | 0.6 | − | − | − | − |
| 0.8 | − | − | − | − | 0.8 | − | − | − | − |
| 1.0 | − | − | − | − | 1.0 | − | − | − | − |
| Adapalene + 0.05 mg/mL 15-OHEPA(<MIC): | | | | | Adapalene + 0.1 mg/mL 15-OHEPA(>MIC) | | | | |
| Adapalene | Growth Index (+++, ++, +, −) | | | | Adapalene | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | +++ | +++ | +++ | +++ | 0.4 | − | − | − | − |
| 0.6 | ++ | +++ | +++ | +++ | 0.6 | − | − | − | − |
| 0.7 | ++ | ++ | ++ | ++ | 0.7 | − | − | − | − |
| 0.8 | ++ | ++ | + | ++ | 0.8 | − | − | − | − |
| Metronidazole + 0.05 mg/mL 15-OHEPA (<MIC): | | | | | Metronidazole + 0.1 mg/mL 15-OHEPA (>MIC): | | | | |
| Metronidazole | Growth Index (+++, ++, +, −) | | | | Metronidazole | Growth Index (+++, ++, +, −) | | | |
| [mg/mL] | Plate 1 | Plate 2 | Plate 3 | Avg | [mg/mL] | Plate 1 | Plate 2 | Plate 3 | Avg |
| 0.2 | +++ | +++ | +++ | +++ | 0.2 | − | − | − | − |
| 0.4 | +++ | +++ | +++ | +++ | 0.4 | − | − | − | − |
| 0.6 | +++ | +++ | +++ | +++ | 0.6 | − | − | − | − |
| 0.8 | +++ | +++ | ++ | +++ | 0.8 | − | − | − | − |
| 1.0 | +++ | ++ | +++ | +++ | 1.0 | − | − | − | − |

15-OHEPA in combination with other compounds including nicotinamide, metranidazole or adapalene did not reduce the growth of P. Acnes below that of 15 OHEPA used alone. However, a spike of 0.05 mg/ml 15-OHEPA to benzoyl peroxide did reduce the MIC of benzoyl peroxide from >0.6, ≦0.8 to >0.4, ≦0.6. These results suggest that 15-OHEPA and benzoyl peroxide may exhibit synergy since 0.05 mg/mL has no effect on its own but when added to benzoyl peroxide it is able to further decrease the growth rate of P. acnes below that of the 15-OHEPA single compound treatment (see, Table 8).

TABLE 8
15-OHEPA Combination Summary

| | | MIC | MIC |
|---|---|---|---|
| | MIC | (mg/mL) with | (mg/mL) with |
| | (mg/mL) as per P- | 0.05 mg/mL | 0.1 mg/mL |
| | 11-0007 or P-11-0008 | 15-OHEPA | 15-OHEPA |
| Compound | Addendum A | spike | spike* |
| Metronidazole | >1 | >1 | <0.2 |
| Nicotinamide | >10, ≦15 | >10, ≦15 | <1 |
| BPO | >0.6, ≦0.8 | >0.4, ≦0.6 | <0.2 |
| Adapalene | >0.8 | >0.8 | <0.2 |
| 15-OHEPA | >0.05, ≦0.075 | N/App | N/App |

*0.1 mg/mL 15-OHEPA is an inhibitory concentration. Complete inhibition was expected for all plates spiked with 0.1 mg/mL.

### Example 3

### Antibacterial Effects of Various Compounds.

The compounds tested in Example 1 were tested to determine their capacity to inhibit the growth of a variety of Gram-negative bacteria, Gram-positive bactgeria, fungi and yeast. In an exemplary method, Mueller Hinton agar plates were streaked with 500 μL of a 10⁵ colony-forming units/mL culture and allowed to dry for at least one minute. A sterile paper disc (6 mm diameter) was then placed in the middle of each plate using sterile forceps. Twenty microliters of a solution of a known concentration of a test compound in filter-sterilized 95% ethanol were pipetted onto each test plate. Two streaked plates served as positive inhibitory controls and contained 6 mm sterile paper discs with standard tetracycline solutions. Two additional plates served as negative inhibition controls and each contained 6 mm sterile paper discs with 20 μL of filter-sterilized 95% ethanol. Two more plates served as positive growth controls and contained no paper disc or added antibacterial agent. The plates were incubated at 32.5±2° C. for 24±4 hours, after which the corrected zone of inhibition (“CZOI”) was determined as follows:

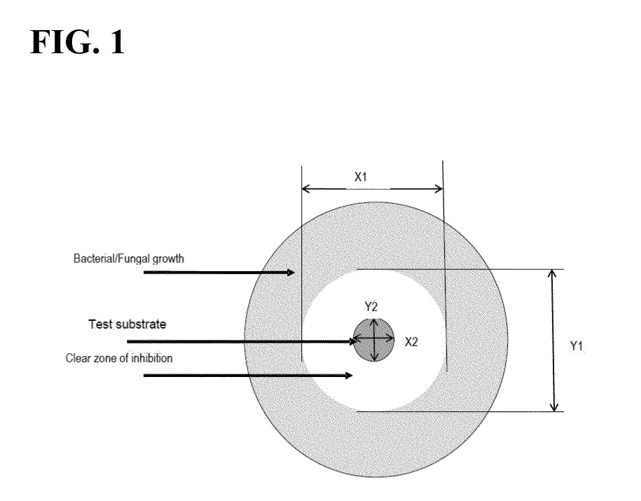

For each plate, the length Y2 and width X2 of the test sample (paper disc) and length Y1 and width X1 of the clear zone were measured (FIG. 1). The CZOI was calculated from these values as shown by the equation below:

$$CZOI = \frac{( X 1 - X 2 ) + ( Y 1 - Y 2 )}{2}$$

Average CZOI measurements for multiple plates are shown in Tables 9-11 below.

TABLE 9
Average CZOI Values for DGLA at various concentrations.

| | | Average CZOI (mm) | | | | |
|---|---|---|---|---|---|---|
| | | 1.4 | 1.2 | 1.0 | 0.8 | 0.6 |
| Organism | Type | mg/mL | mg/mL | mg/mL | mg/mL | mg/mL |
| P. aeroginosa | Gram | 1 | 1 | 1 | 1 | 1 |
| E. coli | negative | 0.25 | 0.5 | 1.5 | 1 | 1 |
| K. pneumoniae | bacteria | 1.25 | 2 | 2.75 | 2 | 1.75 |
| S. aureus | Gram | 2 | 3 | 3 | 3 | 1.25 |
| E. faecalis | positive | 1.75 | 2 | 5 | 3 | 3.25 |
| S. epidermidis | bacteria | 1 | 3 | 6.5 | 3.25 | 4.75 |
| C. striatum | | 2.75 | 4.75 | 2 | 2.75 | 3.75 |
| A. brasiliensis | Fungus | 2.25 | 1.75 | 2.75 | 4 | 1.5 |
| C. albicans | Yeast | 3 | 2 | 1.75 | 0.75 | 1 |

TABLE 10
Average CZOI Values for 15-HETrE at various concentrations.

| | | Average CZOI (mm) | | | | |
|---|---|---|---|---|---|---|
| | | 0.5 | 0.1 | 0.05 | 0.01 | 0.005 |
| Organism | Type | mg/mL | mg/mL | mg/mL | mg/mL | mg/mL |
| P. aeroginosa | Gram | 1 | 1.5 | 1 | 1.5 | 1.5 |
| E. coli | negative | 1 | 1.25 | 1 | 2 | 1.5 |
| K. pneumoniae | bacteria | 1 | 1.5 | 1 | 2 | 2.5 |
| S. aureus | Gram | 1 | 1.75 | 0.75 | 2.5 | 2 |
| E. faecalis | positive | 2.75 | 3.25 | 3.75 | 5.75 | 3.75 |
| S. epidermidis | bacteria | 1.75 | 1 | 1 | 3 | 1.75 |
| C. striatum | | 1.5 | 3.25 | 2.25 | 3.25 | 4 |
| A. brasiliensis | Fungus | 3 | 2.25 | 1.25 | 1 | 1.75 |
| C. albicans | Yeast | 1.25 | 1.5 | 1.25 | 1.75 | 2 |

TABLE 11
Average CZOI Values for 15-OHEPA at various concentrations.

| | | Average CZOI (mm) | | | | |
|---|---|---|---|---|---|---|
| | | 0.1 | 0.075 | 0.05 | 0.025 | 0.010 |
| Organism | Type | mg/mL | mg/mL | mg/mL | mg/mL | mg/mL |
| P. aeroginosa | Gram | 1.5 | 1.75 | 1.5 | 1 | 1.25 |
| E. coli | negative | 1.5 | 1.75 | 1.25 | 1.25 | 2 |
| K. pneumoniae | bacteria | 2.25 | 1.75 | 1 | 1.5 | 2.25 |
| S. aureus | Gram | 2 | 2.25 | 1.25 | 1 | 0 |
| E. faecalis | positive | 1.5 | 1.25 | 2.25 | 1.5 | 2.5 |
| S. epidermidis | bacteria | 1.25 | 3 | 2.25 | 2 | 1 |
| C. striatum | | 1.5 | 2 | 3.5 | 3 | 3.25 |
| A. brasiliensis | Fungus | 2.75 | 3.75 | 2 | 1.75 | 0.25 |
| C. albicans | Yeast | 1 | 2.25 | 2.5 | 1.5 | 1 |

Based on these experiments, a summary of the most effective observed concentrations for each agent per organism is shown in Table 12, and a summary of the lowest concentration for which inhibition was observed is shown in Table 13, below.

TABLE 12
Summary of Most Effective Concentrations Observed.

| | | Most Effective Concentration Observed | | |
|---|---|---|---|---|
| Organism | Type | DGLA | 15-HETrE | 15-OHEPA |
| P. aeroginosa | Gram | 0.6 mg/mL | 0.005 mg/mL | 0.075 mg/mL |
| E. coli | negative | 1.0 mg/mL | 0.01 mg/mL | 0.001 mg/mL |
| K. pneumoniae | bacteria | 1.0 mg/mL | 0.005 mg/mL | 0.001 mg/mL |
| S. aureus | Gram | 0.8 mg/mL | 0.01 mg/mL | 0.075 mg/mL |
| E. faecalis | positive | 1.0 mg/mL | 0.01 mg/mL | 0.001 mg/mL |
| S. epidermidis | bacteria | 1.0 mg/mL | 0.01 mg/mL | 0.075 mg/mL |
| C. striatum | | 1.2 mg/mL | 0.05 mg/mL | 0.05 mg/mL |
| A. brasiliensis | Fungus | 0.8 mg/mL | 0.5 mg/mL | 0.075 mg/mL |
| C. albicans | Yeast | 1.4 mg/mL | 0.005 mg/mL | 0.05 mg/mL |

TABLE 13
Summary of Lowest Concentration With Observed Inhibition.

| | | Lowest Concentration | | |
|---|---|---|---|---|
| | | With Observed Inhibition | | |
| Organism | Type | DGLA | 15-HETrE | 15-OHEPA |
| P. aeroginosa | Gram | 0.6 mg/mL | 0.005 mg/mL | 0.001 mg/mL |
| E. coli | negative | 0.6 mg/mL | 0.005 mg/mL | 0.001 mg/mL |
| K. pneumoniae | bacteria | 0.6 mg/mL | 0.005 mg/mL | 0.001 mg/mL |
| S. aureus | Gram | 0.6 mg/mL | 0.005 mg/mL | 0.025 mg/mL |
| E. faecalis | positive | 0.6 mg/mL | 0.005 mg/mL | 0.001 mg/mL |
| S. epidermidis | bacteria | 0.6 mg/mL | 0.005 mg/mL | 0.001 mg/mL |
| C. striatum | | 0.6 mg/mL | 0.005 mg/mL | 0.001 mg/mL |
| A. brasiliensis | Fungus | 0.6 mg/mL | 0.005 mg/mL | 0.001 mg/mL |
| C. albicans | Yeast | 0.6 mg/mL | 0.005 mg/mL | 0.001 mg/mL |

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

What is claimed is:

1. A composition comprising neomycin sulfate and DGLA.

2. The composition of claim 1, wherein the composition comprises about 0.1 wt. % to about 20 wt. % of DGLA.

3. The composition of claim 2, wherein the DGLA is in free acid form.

4. The composition of claim 1, wherein the DGLA is in a pharmaceutically acceptable ester, derivative, conjugate, or salt form.

5. The composition of claim 4, wherein the DGLA is an alkyl ester of the DGLA.

6. The composition of claim 1, wherein the neomycin sulfate is present in an amount of less than about 3.5 mg/gram of pharmaceutical composition.

7. The composition of claim 1, wherein the composition is suitable for topical delivery.

8. The composition of claim 1, wherein the fatty acid agent has been deodorized prior to use in the composition.

9. The composition of claim 1, wherein the composition inhibits growth of P. aeroginosa, E. coli, K. pneumoniae, S. aureus, E. faecalis, S. epidermidis, C. striatum, A. brasiliensis, C. albicans, or any combination thereof.

10. The composition of claim 1 wherein the composition is an antibacterial composition.

11. The composition of claim 1 wherein the composition is an antifungal composition.

12. A method of treating or preventing a microbial infection comprising administering to a subject in need thereof a composition according to claim 1.

13. The method of claim 12, wherein the composition is topically applied to a wound of the subject.

14. The method of claim 12 wherein the microbial infection is a bacterial infection.

15. The method of claim 12 wherein the microbial infection is a fungal infection.

16. A method of minimizing or preventing scarring comprising administering to a wound of a subject the composition of claim 1.

17. The method of claim 12 wherein the composition further comprises an agent selected from polymyxin B, bacitracin zinc, ampicillin, amoxicillin, imipenem, meropenem, carbapenems, cephalexin, cephalothin, cefalozin, cefuroxime, cefotaxime, ceftazidime, fluoroquinolones, oxazolidinones, lincosamides, metronidazole, clindamycin, erythromycin, quinolone anibiotics, levofloxacin, ciprofloxacin, penicillins, glycopeptides, vancomycin, aminoglycosides, neomycin, gentamicin, tobramycin, trimethoprim/sulfamethoxazole, doxycycline, triclosan, metronidazole, monocycline and tetracycline.
